(19) United States
(12) Patent Application Publication
(10) Pub. No.: US 2008/0113006 A1
(43) Pub. Date: May 15, 2008

(54) Transparent Sheet-Like Cleansing Agent

(75) Inventors: Yoshinobu Saito, Osaka (JP); Daiji Nagahama, Osaka (JP); Takahiro Okuda, Osaka (JP); Tetsuo Nishina, Osaka (JP)

Correspondence Address: NDQ&M WATCHSTONE LLP, 1300 EYE STREET, NW, SUITE 1000 WEST TOWER, WASHINGTON, DC, 20005, US

(73) Assignee: P & PF CO., LTD, Osaka (JP)

(21) Appl. No.: 11/664,693

(22) PCT Filed: Oct. 12, 2004
(86) PCT No.: PCT/JP04/15351

§ 371 Date: Apr. 5, 2007

(51) Int. Cl. A61K 8/36; A61K 8/37; A61K 9/70

(52) U.S. Cl. 424/443; 424/70.22

(57) ABSTRACT

The present invention provides a transparent sheet-like cleansing agent obtained by preparing a composition that contains: (a) a water-soluble high molecular weight compound having a film forming ability; (b) an alkali salt of a fatty acid; and (c) a compound expressed by General Formula (I) below,
General Formula (I)
(where R is a C₄-C₃₄ alkyl group or C₄-C₃₄ alkenyl group, at least one of X₁ and X₂ is —CH₂COOM and the other is a hydrogen atom, where M is an alkali metal, an alkaline-earth metal, ammonium, a lower alkanolamine cation, a lower alkylamine cation, or a basic amino acid cation), and uniformly mixing and dissolving (a), (b), and (c) to prepare an undiluted solution in which a ratio of expansion (volume after mixing and dissolution/volume before mixing and dissolution) caused by bubbles generated during the mixing and dissolution is not greater than 1.6, and performing film-formation from the undiluted solution.

According to the present invention, it is possible to provide a transparent sheet-like cleansing agent that is excellent in transparency, can be formed thin, has good solubility, is excellent in foaming properties, and leaves no residual substance.

### TECHNICAL FIELD

The present invention relates to a sheet-like cleansing agent, and more specifically a transparent sheet-like cleansing agent that is excellent in transparency, can be formed thin, is quickly dissolved, has good foaming properties, and leaves no residual substance.

### BACKGROUND ART

Sheet-like cleansing agents are conventionally used for portable use. For example, as described in JP S57-29520A, a sheet-like cleansing agent is known comprising nonionic cellulose ether, and soap components such as aliphatic soaps and natural soaps.

However, this sheet-like cleansing agent has a problem in that nonionic cellulose ether, serving as an essential component for shaping soap into a sheet, inhibits foaming properties and solubility, and in that a residual substance remains. Furthermore, the conventional sheet-like cleansing agent also has a problem in that a transparent cleansing agent cannot be formed because the strong crystallinity of the soap components makes the cleansing agent opaque. In particular, in the case of the conventional sheet-like cleansing agent comprising a film forming agent and soap components, a sheet-like cleansing agent with improved solubility in use is produced using a method in which when mixing and dissolving a composition for preparing an undiluted solution for film-formation, the composition is dissolved with agitation such that bubbles are generated in the undiluted solution, and then film-formation is performed from the foamed undiluted solution. Therefore, the conventional sheet-like cleansing agent cannot be made thin, in addition to the fact that it cannot be made transparent.

Furthermore, as described in JP H2-22400A, a sheet-like cleansing agent is also known produced using a method in which soft paper is impregnated with a soap aqueous solution, or an aqueous solution obtained by adding an appropriate amount of sodium alkyl ether sulfate, ammonium alkyl ether sulfate, or fatty acid alkanolamide to a surfactant, and then the soft paper is dried and cut into predetermined shape and size.

However, the sheet-like cleansing agent has a problem in that it is hard to use because soft paper remains when the cleansing agent is used, and in that foaming properties are poor. Furthermore, since soft paper is contained, there is a problem in that a transparent cleansing agent cannot be formed and in that the thickness cannot be made thin.

A sheet-like cleansing agent is used in an amount of one sheet for one time, and the amount used for one time is limited to be smaller than that of commonly used solid soaps. In this case, the sheet-like cleansing agent cannot be used in practice unless the cleansing agent is quickly dissolved and has extremely good foaming properties. Even if there is no problem regarding the practicability, it is impossible to improve portability, which is an advantage of sheet-like cleansing agents, unless the thickness is made thin. Furthermore, it is impossible to improve the degree of freedom in design for forming a product that is excellent in design, unless a transparent cleansing agent cannot be formed.

The present invention has been made in order to address the problems described above, and it is an object thereof to provide a transparent sheet-like cleansing agent that is excellent in transparency, can be formed thin, has good solubility, is excellent in foaming properties, and leaves no residual substance.

### DISCLOSURE OF INVENTION

The inventors conducted in-depth research on the problems described above, and found that a transparent sheet-like cleansing agent that is excellent in transparency, can be formed thin, has improved solubility and foaming properties, and leaves no residual substance can be provided by uniformly mixing and dissolving a composition that contains a water-soluble high molecular weight compound having a film forming ability, an alkali salt of a fatty acid, and a specific surfactant to prepare an undiluted solution in which the ratio of expansion (volume after mixing and dissolution/volume of undiluted solution) caused by bubbles generated during the mixing and dissolution is 1.6 or less, and performing film-formation from the undiluted solution, and as a result, the present invention has been achieved.

More specifically, the present invention is directed to a transparent sheet-like cleansing agent obtained by

preparing a composition that contains:
(a) a water-soluble high molecular weight compound having a film forming ability;
(b) an alkali salt of a fatty acid; and
(c) a compound expressed by General Formula (I) below,
General Formula (I)
(where R is a C₄-C₃₄ alkyl group or C₄-C₃₄ alkenyl group, at least one of X₁ and X₂ is —CH₂COOM and the other is a hydrogen atom, where M is an alkali metal, an alkaline-earth metal, ammonium, a lower alkanolamine cation, a lower alkylamine cation, or a basic amino acid cation), and

uniformly mixing and dissolving (a), (b), and (c) to prepare an undiluted solution in which a ratio of expansion (volume after mixing and dissolution/volume before mixing and dissolution) caused by bubbles generated during the mixing and dissolution is not greater than 1.6, and performing film-formation from the undiluted solution.

As a preferred embodiment of the present invention, it is also possible that the transparent sheet-like cleansing agent has an expansion ratio of 1 and a thickness of 20 to 180 μm. Furthermore, it is also possible that the undiluted solution having an expansion ratio of not greater than 1.6 is obtained, by preparing an aqueous solution in which at least (a) has been dissolved in an excessive amount of water and an aqueous solution in which at least (b) and (c) have been dissolved in an excessive amount of water, mixing and dissolving the aqueous solutions, and then drying the mixture to a predetermined water content for film-formation.

As a preferred embodiment, it is also possible that the compound (c) is sodium salt of dodecane-1,2-diol carboxymethylate.

As a preferred embodiment, it is also possible that the water-soluble high molecular weight compound (a) having the film forming ability is at least one selected from cellulose ethers.

As a preferred embodiment of the present invention, it is also possible that a content of the compound (c) is 2 to 25 wt %, that a content of the water-soluble high molecular weight compound (a) having the film forming ability is 15 to 45 wt %, or that a content of the alkali salt (b) of the fatty acid is 35 to 65 wt %.

### BEST MODE FOR CARRYING OUT THE INVENTION

Hereinafter, the present invention is described in detail. A transparent sheet-like cleansing agent of the present invention is obtained by preparing a composition that contains: (a) a water-soluble high molecular weight compound having a film forming ability; (b) an alkali salt of a fatty acid; and (c) a compound expressed by General Formula (I) below,
General Formula (I)

(where R is a C₄-C₃₄ alkyl group or C₄-C₃₄ alkenyl group, at least one of X₁ and X₂ is —CH₂COOM and the other is a hydrogen atom, where M is an alkali metal, an alkaline-earth metal, ammonium, a lower alkanolamine cation, a lower alkylamine cation, or a basic amino acid cation), and uniformly mixing and dissolving (a), (b), and (c) to prepare an undiluted solution in which the ratio of expansion (volume after mixing and dissolution/volume before mixing and dissolution) caused by bubbles generated during the mixing and dissolution is 1.6 or less, and performing film-formation from the undiluted solution.

In the present invention, there is no specific limitation regarding the water-soluble high molecular weight compound (a) having the film forming ability, as long as the compound is water-soluble and can be used for forming a sheet-like film. Examples thereof include: celluloses such as cellulose, and cellulose ether (methylcellulose, hydroxyethylmethylcellulose, hydroxypropylmethylcellulose, hydroxybutylmethylcellulose, hydroxyethylcellulose, hydroxypropylcellulose, hydroxybutylcellulose, sodium carboxymethylcellulose, for example); polyvinyl alcohol; and polysaccharides such as pullulan. Of these, cellulose ethers, in particular, hydroxypropylmethylcellulose and hydroxypropylcellulose are preferable because they are flexible and strong. It should be noted that the water-soluble high molecular weight compounds having the film forming ability may be used alone or in combination of two or more.

In the composition of the transparent sheet-like cleansing agent, the content of the water-soluble high molecular weight compound (a) having the film forming ability is preferably 15 to 45 wt %, and particularly preferably 20 to 35 wt %. If the content is too small, then the cleansing agent cannot be shaped into a sheet. On the contrary, if the content is too large, then the water-soluble high molecular weight compound (a) makes the cleansing agent in the form of paste when it is used, which is not preferable because the foaming properties may be inhibited or a residual substance may remain.

In the present invention, the fatty acid used for the alkali salt (b) of the fatty acid is a linear or branched, and saturated or unsaturated fatty acid having 8 to 24 carbon atoms, and preferably 12 to 18 carbon atoms. Specific preferable examples of the fatty acid include: saturated fatty acids such as lauric acid, myristic acid, palmitic acid, stearic acid, and isostearic acid; unsaturated fatty acids such as oleic acid; and their mixtures such as coconut oil fatty acid, palm oil fatty acid, palm kernel oil fatty acid, beef tallow fatty acid, and hardened beef tallow fatty acid.

Examples of the alkali in the alkali salt (b) of the fatty acid include: alkali metals such as sodium, potassium, and lithium; alkaline-earth metals such as calcium and magnesium; and organic amine such as ethanolamine (monoethanolamine, diethanolamine, and triethanolamine), and basic amino acid (lysine, arginine, and histidine, for example). As the alkali, sodium and potassium are preferably used.

It should be noted that the alkali salts of the fatty acids may be used alone or in combination of two or more.

In the composition of the transparent sheet-like cleansing agent, the content of the alkali salt (b) of the fatty acid is preferably 35 to 65 wt %, and particularly preferably 40 to 60 wt %. If the content is too small, then solidifying properties are poor. On the contrary, if the content is too large, then the sheet-like cleansing agent is hard and brittle, which is not preferable.

In the compound (c) of General Formula (I) above used in the present invention, the C₄-C₃₄ alkyl group as R is a linear or branched alkyl group, and examples thereof include a butyl group, hexyl group, octyl group, decyl group, dodecyl group, tetradecyl group, hexadecyl group, octadecyl group, eicosyl group, docosyl group, tetracosyl group, 2-ethylhexyl group, 2-hexyldecyl group, 2-octylundecyl group, 2-decyltetradecyl group, and 2-undecylhexadecyl group. The C₄-C₃₄ alkenyl group is a linear or branched alkenyl group, and examples thereof include an octenyl group, decenyl group, dodecenyl group, tetradecenyl group, hexadecenyl group, and octadecenyl group. In General Formula (I) above, R is preferably a C₈-C₁₈ linear alkyl group, and particularly preferably a decyl group or dodecyl group, because they provide the sheet-like cleansing agent with particularly good foaming properties.

In General Formula (I) above, examples of the alkali metal as M include sodium, potassium, and lithium. Examples of the alkaline-earth metal include calcium, magnesium, and barium. Examples of lower alkanolamine constituting the lower alkanolamine cation include alkanolamine having 1 to 4 carbon atoms, such as ethanolamine, n-propanolamine, and isopropanolamine, and any one of monoalkanolamine, dialkanolamine, and trialkanolamine may be used. Examples of lower alkylamine constituting the lower alkylamine cation include alkylamine having 1 to 4 carbon atoms, such as methylamine, ethylamine, n-propylamine, and isopropylamine, and any one of monoalkylamine, dialkylamine, and trialkylamine may be used. Examples of a basic amino acid constituting the basic amino acid include lysine, arginine, ornithine, and histidine. In General Formula (I) above, M is preferably an alkali metal or lower alkanolamine cation, and particularly preferably sodium or potassium, because they provide the transparent sheet-like cleansing agent with particularly good foaming properties.

In General Formula (I) above, at least one of X₁ and X₂ is —CH₂COOM, and the other is a hydrogen atom. The solubility and the foaming properties of the transparent sheet-like cleansing agent are improved by blending the thus structured compound (c) therewith.

Specific examples of the compound (c) include sodium salt of octane-1,2-diol carboxymethylate, sodium salt of decane-1,2-diol carboxymethylate, sodium salt of dodecane-1,2-diol carboxymethylate, sodium salt of tetradecane-1,2-diol carboxymethylate, sodium salt of hexadecane-1,2-diol carboxymethylate, and sodium salt of octadecane-1,2-diol carboxymethylate.

The compound (c) may be a compound in which either one of X₁ and X₂ is —CH₂COOM, may be a compound in which both of X₁ and X₂ are —CH₂COOM, or may be their mixture. Furthermore, the compounds (c) may be used alone or in combination of two or more.

In a case where M is sodium, the compound (c) can be obtained by carboxymethylation of alkyl-1,2-diol in an ether-based solvent such as dioxane, using metal sodium or the like in a nitrogen gas stream, and then reacting the resultant with sodium chloroacetate.

In the composition of the transparent sheet-like cleansing agent, the content of the compound (c) is preferably 2 to 25 wt %, and particularly preferably 3 to 15 wt %. If the content is too small, then solubility and foaming properties are not sufficient, and thus the sheet-like cleansing agent cannot be used in practice. On the contrary, if the content is too large, then the cleansing agent becomes sticky, which is not preferable because the film forming workability may be deteriorated.

The composition of the transparent sheet-like cleansing agent of the present invention contains, as essential components, the water-soluble high molecular weight compound (a) having the film forming ability, the alkali salt (b) of the fatty acid, and the compound (c) of General Formula (I) above. However, if necessary, the composition may further contain additives that are commonly blended with the transparent sheet-like cleansing agent, within the range not interfering with the effects of the present invention. Examples thereof include: anionic surfactants such as N-long-chain acylamino acid salt, alkyl sulfate salt, and polyoxyethylene alkyl ether sulfate; amphoteric surfactants such as imidazoline-based amphoteric surfactant and betaine-based amphoteric surfactant; nonionic surfactants such as polyoxyethylene alkyl ether, polyoxyethylene fatty acid ester, sucrose fatty acid ester, alkyl glycoside, and maltitol hydroxy aliphatic ether; cationic surfactants such as trimethyl alkyl ammonium chloride; moisturizing agents such as glycerin, diglycerin, 1,3-butylene glycol, propylene glycol, dipropylene glycol, polyethylene glycol, sucrose, sorbit, and sodium hyaluronate; chelating agents such as edetate; components extracted from plants such as Japanese swertia, peony, iris, field horsetail, aloe, chamomile, eucalyptus oil, and dipotassium glycyrrhetinate; agents such as tranexamic acid and arbutin; aroma chemicals; dyes; and antiseptic agents.

Next, a method for producing the transparent sheet-like cleansing agent of the present invention is described.

The transparent sheet-like cleansing agent of the present invention is produced as below. First, the water-soluble high molecular weight compound (a) having the film forming ability, the alkali salt (b) of the fatty acid, and the compound (c) of General Formula (I) above, and, if necessary, additives are dissolved in a predetermined ratio in water, so that an undiluted solution with the components uniformly mixed and dissolved is prepared. At that time, the materials are dissolved with agitation using an agitator such as a homomixer rotating at a high speed, and thus air is contained in the aqueous solution, so that the volume of the undiluted solution after mixing and dissolution becomes larger than that of the undiluted solution before mixing and dissolution. When film-formation is performed from this undiluted solution without any processing, due to air contained in the aqueous solution, the strength of a formed film is lowered and the sheet-like cleansing agent is not provided with transparency. Thus, film-formation is performed after the ratio (volume after mixing and dissolution/volume of undiluted solution) of the undiluted solution expanded with bubbles generated during the mixing and dissolution is made 1.6 or less.

Regarding methods for making the expansion ratio of the undiluted solution after mixing and dissolution 1.6 or less, the predetermined expansion ratio may be obtained, for example, using a method in which the compounds are dissolved while the rotating speed of the agitator and the like are controlled such that the expansion ratio does not exceed a predetermined value during the mixing and dissolution, a method in which the undiluted solution after mixing and dissolution is placed still for deaeration, or a method in which after the compounds are mixed and dissolved in an excessive amount of water, water is vaporized to obtain an undiluted solution with a predetermined water content for film-formation.

Furthermore, an undiluted solution with a predetermined expansion ratio may be obtained using a method in which an aqueous solution is prepared by heating and dissolving the components other than the water-soluble high molecular weight compound (a) having the film forming ability in an excessive amount of water, the aqueous solution is mixed and dissolved in post-treatment with an aqueous solution in which the water-soluble high molecular weight compound (a) having the film forming ability has been dissolved in an excessive amount of water, and then the mixture is dried to a predetermined water content for film-formation. More specifically, heating and dissolution in an excessive amount of water allows mixing and dissolution to be performed without generating much bubbles, but the water-soluble high molecular weight compound (a) having the film forming ability cannot be heated so much. Accordingly, an undiluted solution with the components mixed and dissolved can be prepared without generating much bubbles using a method in which an aqueous solution is prepared by heating and dissolving the components other than the water-soluble high molecular weight compound (a) having the film forming ability in an excessive amount of water, then the aqueous solution is mixed and dissolved with an aqueous solution in which the water-soluble high molecular weight compound (a) having the film forming ability has been dissolved in an excessive amount of water, and water is vaporized to a predetermined water content for film-formation, at a temperature lower than the temperature at which the aqueous solution with the components mixed and dissolved starts to be formed into a film. This method is particularly effective when preparing an undiluted solution having an expansion ratio of 1.2 or less.

In a case of mixing and dissolution in an excessive amount of water, the amount of the excessive water for mixing and dissolution is determined as appropriate, for example, based on the components of the composition, the expansion ratio of the undiluted solution, and the thickness of the sheet-like cleansing agent that is to be formed.

After preparing an undiluted solution in which the composition of the transparent sheet-like cleansing agent has been uniformly mixed and dissolved in this manner, film-formation can be performed using conventionally known methods. Film-formation is formed, for example, using a method in which the prepared undiluted solution is supplied to a surface of a base heated to approximately 60 to 90° C. so that the undiluted solution flows to be extended and is dried thereon. At that time, the undiluted solution flows to be extended preferably such that the thickness after drying is 20 to 180 μm.

According to the thus formed transparent sheet-like cleansing agent of the present invention, it is possible to reduce bubbles contained in the formed sheet-like cleansing agent by uniformly mixing and dissolving the composition of the transparent sheet-like cleansing agent to prepare an undiluted solution in which the ratio of expansion (volume after mixing and dissolution/volume of undiluted solution) caused by bubbles generated during the mixing and dissolution is 1.6 or less, and performing film-formation from the undiluted solution. Thus, the transparency of the sheet-like cleansing agent can be improved, and the strength can be prevented from being lowered by bubbles, so that a thin sheet-like cleansing agent can be formed. Moreover, since a thin sheet-like cleansing agent is formed by reducing bubbles contained in the formed sheet-like cleansing agent, the detergency can be kept without being lowered.

Furthermore, when the compound (c) of General Formula (I) above in the composition is blended, the Krafft point of the composition is lowered, and thus the solubility is improved. Also, excellent foaming properties of the compound (c) improve the foaming properties of the composition. Accordingly, not only that the sheet-like cleansing agent has transparency and is thin, it is quickly dissolved, has good foaming properties, and leaves no residual substance. Thus, the sheet-like cleansing agent is excellent in design, portability, and practicability.

### EXAMPLES

Hereinafter, the present invention is described more specifically by way of examples, but the present invention is not limited to these examples.

### Examples 1 to 28 and Comparatives Example 1 to 11

Transparent sheet-like cleansing agents were prepared using the following method, with components listed in Tables 1 to 6. In Tables 1 to 6, the amount of the components are expressed by parts by weight.

Hydroxypropylethylcellulose was uniformly dissolved in ion exchanged water. At that time, the ion exchanged water was used in an amount larger than that for the water content for film-formation, the amount being four times as large as the amount in parts by weight of the hydroxypropylethylcellulose.

The components other than the hydroxypropylethylcellulose were uniformly mixed and dissolved in ion exchanged water at a temperature of 70° C. At that time, the ion exchanged water was used in an amount larger than that for the water content for film-formation, the amount being four times as large as the amount in parts by weight of the components other than the hydroxypropylethylcellulose.

Then, the aqueous solutions were uniformly mixed and dissolved at a temperature of 50° C., and dried to a predetermined water content for film-formation, and thus an undiluted solution was obtained.

The thus obtained undiluted solution was poured onto a rotating drum heated to 70° C., dried at 70° C. for two hours, and then cut, and thus substantially anhydrous sheet-like cleansing agents having a size of 7×5 cm and predetermined thicknesses listed in Tables 1 to 6 were obtained.

It should be noted that in Examples 14 to 16 and Comparative Examples 6 to 7, the expansion ratio was obtained by the following equation.

Expansion ratio=(B)/(A)

An undiluted solution in an amount of 500 liters was produced using a production vessel having a capacity of 1000 liters, and the height (A) of the liquid level at that time was measured. Using a homomixer at a rotational speed of 3000 rpm, bubbles were generated in the undiluted solution, and the undiluted solution was further mixed and agitated. Then, the height (B) of the liquid level was measured, and the expansion ratio was obtained as (B)/(A).

### Comparative Examples 12 to 16

Sheet-like cleansing agents were prepared as in Example 1, except that surfactants listed in Table 6 were used instead of sodium salt of dodecane-1,2-diol carboxymethylate used in Example 1.

TABLE 1

| | | | | | | | Com. | Com. |
|---|---|---|---|---|---|---|---|---|
| | Ex. 1 | Ex. 2 | Ex. 3 | Ex. 4 | Ex. 5 | Ex. 6 | Ex. 1 | Ex. 2 |
| sodium laurate | 53 | 50 | 45 | 40 | 35 | 30 | 50 | 25 |
| sodium salt of | 2 | 5 | 10 | 15 | 20 | 25 | 0 | 30 |
| dodecane-1,2-diol | | | | | | | | |
| carboxymethylate | | | | | | | | |
| hydroxypropylethylcellulose | 25 | 25 | 25 | 25 | 25 | 25 | 25 | 25 |
| glycerin | 15 | 15 | 15 | 15 | 15 | 15 | 20 | 15 |
| water | 5 | 5 | 5 | 5 | 5 | 5 | 5 | 5 |
| total | 100 | 100 | 100 | 100 | 100 | 100 | 100 | 100 |
| thickness | 80μ | 80μ | 80μ | 80μ | 80μ | 80μ | 80μ | 80μ |
| expansion ratio | 1 | 1 | 1 | 1 | 1 | 1 | 1 | 1 |
| transparency | B | A | A | A | A | B | D | C |
| foaming properties | B | A | A | A | A | A | D | C |
| solubility | B | A | A | A | A | B | D | C |
| film forming workability | B | A | A | A | A | B | D | C |

TABLE 2

| | | | | Ex. | Ex. | Ex. | Com. | Com. | Com. |
|---|---|---|---|---|---|---|---|---|---|
| | Ex. 7 | Ex. 8 | Ex. 9 | 10 | 11 | 12 | Ex. 3 | Ex. 4 | Ex. 5 |
| sodium laurate | 45 | 45 | 45 | 45 | 45 | 45 | 45 | 45 | 45 |
| sodium salt of | 10 | 10 | 10 | 10 | 10 | 10 | 10 | 10 | 10 |
| dodecane-1,2-diol | | | | | | | | | |
| carboxymethylate | | | | | | | | | |
| hydroxypropylethylcellulose | 25 | 25 | 25 | 25 | 25 | 25 | 25 | 25 | 25 |
| glycerin | 15 | 15 | 15 | 15 | 15 | 15 | 15 | 15 | 15 |
| water | 5 | 5 | 5 | 5 | 5 | 5 | 5 | 5 | 5 |
| total | 100 | 100 | 100 | 100 | 100 | 100 | 100 | 100 | 100 |
| thickness | 20μ | 40μ | 70μ | 100μ | 120μ | 180μ | 10μ | 15μ | 190μ |
| expansion ratio | 1 | 1 | 1 | 1 | 1 | 1 | 1 | 1 | 1 |
| transparency | A | A | A | A | A | B | B | B | C |
| foaming properties | A | A | A | A | A | A | D | C | C |
| solubility | A | A | A | A | A | B | D | C | C |
| film forming workability | B | A | A | A | A | B | D | C | C |

TABLE 3

| | Ex. | Ex. | Ex. | Ex. | Com. | Com. |
|---|---|---|---|---|---|---|
| | 13 | 14 | 15 | 16 | Ex. 6 | Ex. 7 |
| sodium laurate | 45 | 45 | 45 | 45 | 45 | 45 |
| sodium salt of | 10 | 10 | 10 | 10 | 10 | 10 |
| dodecane-1,2-diol | | | | | | |
| carboxymethylate | | | | | | |
| hydroxypropylethylcellulose | 25 | 25 | 25 | 25 | 25 | 25 |
| glycerin | 15 | 15 | 15 | 15 | 15 | 15 |
| water | 5 | 5 | 5 | 5 | 5 | 5 |
| total | 100 | 100 | 100 | 100 | 100 | 100 |
| thickness | 60μ | 60μ | 60μ | 60μ | 60μ | 60μ |
| expansion ratio | 1 | 1.2 | 1.4 | 1.6 | 1.8 | 2 |
| transparency | A | A | B | B | C | D |
| foaming properties | A | A | A | A | C | C |
| solubility | A | A | A | A | A | A |
| film forming workability | A | A | A | A | C | D |

TABLE 4

| | Ex. | Ex. | Ex. | Ex. | Ex. | Ex. | Ex. | Com. | Com. |
|---|---|---|---|---|---|---|---|---|---|
| | 17 | 18 | 19 | 20 | 21 | 22 | 23 | Ex. 8 | Ex. 9 |
| sodium laurate | 45 | 45 | 45 | 45 | 45 | 45 | 40 | 45 | 35 |
| sodium salt of | 10 | 10 | 10 | 10 | 10 | 10 | 10 | 10 | 10 |
| dodecane-1,2-diol | | | | | | | | | |
| carboxymethylate | | | | | | | | | |
| hydroxypropylethylcellulose | 15 | 20 | 25 | 30 | 35 | 40 | 45 | 10 | 50 |
| glycerin | 25 | 20 | 15 | 10 | 5 | 0 | 0 | 30 | 0 |
| water | 5 | 5 | 5 | 5 | 5 | 5 | 5 | 5 | 5 |
| total | 100 | 100 | 100 | 100 | 100 | 100 | 100 | 100 | 100 |
| thickness | 70μ | 70μ | 70μ | 70μ | 70μ | 70μ | 70μ | 70μ | 70μ |
| expansion ratio | 1 | 1 | 1 | 1 | 1 | 1 | 1 | 1 | 1 |
| transparency | A | A | A | A | A | B | B | B | C |
| foaming properties | A | A | A | A | A | B | B | C | C |
| solubility | A | A | A | A | A | B | B | C | C |
| film forming workability | B | A | A | A | A | B | B | D | D |

TABLE 5

| | | | | | Com. | Com. |
|---|---|---|---|---|---|---|
| | Ex. | Ex. | Ex. | Ex. | Ex. | Ex. |
| | 24 | 25 | 26 | 27 | 10 | 11 |
| sodium myristate | 35 | 45 | 55 | 65 | 25 | 75 |
| sodium salt of | 3 | 13 | 13 | 5 | 13 | — |
| dodecane-1,2-diol | | | | | | |
| carboxymethylate | | | | | | |
| hydroxypropylethylcellulose | 20 | 20 | 20 | 20 | 20 | 20 |
| glycerin | 37 | 17 | 7 | 5 | 37 | 0 |
| water | 5 | 5 | 5 | 5 | 5 | 5 |
| total | 100 | 100 | 100 | 100 | 100 | 100 |
| thickness | 55μ | 55μ | 55μ | 55μ | 55μ | 55μ |
| expansion ratio | 1 | 1 | 1 | 1 | 1 | 1 |
| transparency | B | A | A | B | B | D |
| foaming properties | B | A | A | B | C | D |
| solubility | B | A | A | B | C | D |
| film forming workability | B | A | A | B | C | D |

TABLE 6

| | | Com. | Com. | Com. | Com. | Com. |
|---|---|---|---|---|---|---|
| | Ex. 28 | Ex. 12 | Ex. 13 | Ex. 14 | Ex. 15 | Ex. 16 |
| sodium laurate | 35 | — | 35 | 35 | 35 | 35 |
| sodium myristate | 5 | — | 5 | 5 | 5 | 5 |
| sodium palmitate | 3 | — | 3 | 3 | 3 | 3 |
| sodium stearate | 2 | — | 2 | 2 | 2 | 2 |
| sodium salt of | 10 | — | — | — | — | — |
| dodecane-1,2-diol | | | | | | |
| carboxymethylate | | | | | | |
| sodium lauryl sulfate | — | 55 | 10 | — | — | — |
| coconut oil fatty acid | — | — | — | 10 | — | — |
| acylmethyl taurine | | | | | | |
| coconut oil fatty acid | — | — | — | — | 10 | — |
| amidopropyl betaine | | | | | | |
| lauryl imidazolinium betaine | — | — | — | — | — | 10 |
| hydroxypropylethylcellulose | 25 | 25 | 25 | 25 | 25 | 25 |
| glycerin | 15 | 15 | 15 | 15 | 15 | 15 |
| water | 5 | 5 | 5 | 5 | 5 | 5 |
| total | 100 | 100 | 100 | 100 | 100 | 100 |
| thickness | 60μ | 60μ | 60μ | 60μ | 60μ | 60μ |
| expansion ratio | 1 | 1 | 1 | 1 | 1 | 1 |
| transparency | A | D | D | D | D | D |
| foaming properties | A | D | C | C | C | C |
| solubility | A | D | C | C | C | C |
| film forming workability | A | D | C | C | C | C |

The following evaluation tests were conducted on the sheet-like cleansing agents obtained in Examples 1 to 28 and Comparative Examples 1 to 16. The results are shown in Tables 1 to 6.

<Evaluation Methods>

1. Transparency

The transparency was evaluated by visual observation based on the following criteria.

A: extremely transparent

B: transparent

C: semitransparent

D: opaque

2. Foaming Properties Using artificial hard water containing 70 ppm of calcium carbonate, 1% aqueous solutions of the samples were prepared. Then, the 1% aqueous solutions were agitated at 40° C. for a predetermined time with a mixer at a predetermined rotational speed. The amount of bubbles generated was measured. From the amount of bubbles measured, the foaming properties were evaluated based on the following criteria.

A: 2200 ml or more

B: 2000 ml or more and less than 2200 ml

C: 1800 ml or more and less than 2000 ml

D: less than 1800 ml

3. Solubility

The samples having a size of 7×5 cm and a thickness of approximately 100 μm were placed on palms of 10 specialized test people's hands, and were used after being dissolved with tap water (25° C.). The solubility was evaluated based on the following criteria.

A: 8 or more specialized test people confirmed that the solubility is good and there is no residual substance.

B: 6 or more and less than 8 specialized test people confirmed that the solubility is good and there is no residual substance.

C: 3 or more and less than 6 specialized test people confirmed that the solubility is good and there is no residual substance.

D: less than 3 specialized test people confirmed that the solubility is good and there is no residual substance.

4. Film Forming Workability

The film forming workability was evaluated based on the following criteria, by comprehensively considering the state in which the sample films were formed, regarding whether or not the viscosity was appropriate and there was no unevenness in the thickness, whether or not the peelability from the rotating drum was good, and whether or not the film properties were good.

A: film forming workability is extremely good.

B: film forming workability is good.

C: film forming workability is comparatively poor.

D: film forming workability is poor.

Tables 1 to 6 show that the transparent sheet-like cleansing agents of Examples 1 to 28 are excellent in all of transparency, foaming properties, solubility, and film forming workability. On the other hand, the sheet-like cleansing agents of Comparative Examples 6 and 7 in which the expansion ratio was more than 1.6, those of Comparative Examples 1 and 11 in which sodium salt of dodecane-1,2-diol carboxymethylate was not blended, and those of Comparative Examples 12 to 16 in which other surfactants were used instead of sodium salt of dodecane-1,2-diol carboxymethylate are poor at least in any one of transparency, foaming properties, solubility, and film forming workability.

### Example 29

Sodium salt of octane-1,2-diol carboxymethylate was used instead of sodium salt of dodecane-1,2-diol carboxymethylate used in Examples 1 to 28, and similar evaluation tests were conducted. As a result, A or B was obtained in all evaluation items.

As is clear from the description above, according to the transparent sheet-like cleansing agent of the present invention, it is possible to reduce bubbles contained in the formed sheet-like cleansing agent by uniformly mixing and dissolving the composition of the transparent sheet-like cleansing agent to prepare an undiluted solution in which the ratio of expansion (volume after mixing and dissolution/volume of undiluted solution) caused by bubbles generated during the mixing and dissolution is 1.6 or less, and performing film-formation from the undiluted solution. Thus, the transparency of the sheet-like cleansing agent can be improved, and the transparency can be kept even when a thick sheet-like cleansing agent is formed. Furthermore, the strength can be prevented from being lowered by bubbles, so that a thin sheet-like cleansing agent can be formed. Moreover, since a thin sheet-like cleansing agent is formed by reducing bubbles contained in the formed sheet-like cleansing agent, the detergency can be kept without being lowered.

Furthermore, when the compound (c) of General Formula (I) above in the composition is blended, the Krafft point of the composition is lowered, and thus the solubility is improved. Also, excellent foaming properties of the compound (c) improve the foaming properties of the composition. Accordingly, not only that the sheet-like cleansing agent has transparency and is thin, it is quickly dissolved, has good foaming properties, and leaves no residual substance. Thus, the sheet-like cleansing agent is excellent in design, portability, and practicability.

### INDUSTRIAL APPLICABILITY

The transparent sheet-like cleansing agent of the present invention is excellent in transparency, can be formed thin, is quickly dissolved, has good foaming properties, and leaves no residual substance. Thus, it is used as a cleansing agent that is excellent in design, portability, practicability, and the like.

1. A transparent sheet-like cleansing agent obtained by
preparing a composition that contains:
(a) a water-soluble high molecular weight compound having a film forming ability;
(b) an alkali salt of a fatty acid; and
(c) a compound expressed by General Formula (I) below,
General Formula (I)
(where R is a C₄-C₃₄ alkyl group or C₄-C₃₄ alkenyl group, at least one of X₁ and X₂ is —CH₂COOM and the other is a hydrogen atom, where M is an alkali metal, an alkaline-earth metal, ammonium, a lower alkanolamine cation, a lower alkylamine cation, or a basic amino acid cation), and
uniformly mixing and dissolving (a), (b), and (c) to prepare an undiluted solution in which a ratio of expansion (volume after mixing and dissolution/volume before mixing and dissolution) caused by bubbles generated during the mixing and dissolution is not greater than 1.6, and performing film-formation from the undiluted solution.

2. The transparent sheet-like cleansing agent according to claim 1,
wherein the expansion ratio is 1 and a thickness is 20 to 180 μm.

3. The transparent sheet-like cleansing agent according to claim 1,
wherein the undiluted solution having an expansion ratio of not greater than 1.6 is obtained, by preparing an aqueous solution in which at least (a) has been dissolved in an excessive amount of water and an aqueous solution in which at least (b) and (c) have been dissolved in an excessive amount of water, mixing and dissolving the aqueous solutions, and then drying the mixture to a predetermined water content for film-formation.

4. The transparent sheet-like cleansing agent according to claim 1,
wherein the compound (c) is sodium salt of dodecane-1,2-diol carboxymethylate.

5. The transparent sheet-like cleansing agent according to claim 1,
wherein the water-soluble high molecular weight compound (a) having the film forming ability is at least one selected from cellulose ethers.

6. The transparent sheet-like cleansing agent according to claim 1,
wherein a content of the compound (c) is 2 to 25 wt %.

7. The transparent sheet-like cleansing agent according to claim 1,
wherein a content of the water-soluble high molecular weight compound (a) having the film forming ability is 15 to 45 wt %.

8. The transparent sheet-like cleansing agent according to claim 1,
wherein a content of the alkali salt (b) of the fatty acid is 35 to 65 wt %.
